(19) United States
(12) Patent Application Publication
(10) Pub. No.: US 2020/0037881 A1
(43) Pub. Date: Feb. 6, 2020

(54) METHOD, SYSTEM, AND NON-TRANSITORY COMPUTER-READABLE RECORDING MEDIUM FOR CONTROLLING MONITORING DEVICE INCLUDING PLURALITY OF LIGHT EMISSION UNITS AND PLURALITY OF LIGHT RECEPTION UNITS

(71) Applicant: OBE LAB., INC., Daejeon (KR)

(72) Inventors: Jong Kwan CHOI, Seoul (KR); Il Taek HAN, Yongin-si (KR); Dae Leem KWON, Seoul (KR)

(73) Assignee: OBE LAB., INC., Daejeon (KR)

(21) Appl. No.: 16/601,663

(22) Filed: Oct. 15, 2019

Related U.S. Application Data
(63) Continuation of application No. PCT/KR2018/001713, filed on Feb. 8, 2018.

(30) Foreign Application Priority Data
Jan. 31, 2018 (KR) 10-2018-0012197

(51) Int. Cl. A61B 5/00

(52) U.S. Cl. CPC A61B 5/0075; A61B 5/0082; A61B 5/0068

(57) ABSTRACT

According to one aspect of the invention, there is provided a method for controlling a monitoring device including a plurality of light emission units (or sources) and a plurality of light reception units (or detectors), comprising the steps of: causing the plurality of light emission units to sequentially generate optical signals in a mutually exclusive manner according to a time division scheme; and with reference to a distance between an m-th light emission unit of the plurality of light emission units that generates an optical signal to be measured and an n-th light reception unit of the plurality of light reception units that detects the optical signal to be measured, dynamically controlling a measurement circuit gain that the n-th light reception unit uses to detect the optical signal generated by the m-th light emission unit.

### CROSS-REFERENCE TO RELATED APPLICATION(S)

This application is a continuation application of Patent Cooperation Treaty (PCT) international application Serial No. PCT/KR2018/001713, filed on Feb. 8, 2018, which claims priority to Korean Patent Application Serial No. 10-2018-0012197, filed on Jan. 31, 2018. The entire contents of PCT international application Serial No. PCT/KR2018/001713 and Korean Patent Application Serial No. 10-2018-0012197 are hereby incorporated by reference.

### FIELD OF THE INVENTION

The present invention relates to a method, system, and non-transitory computer-readable recording medium for controlling a monitoring device including a plurality of light emission units and a plurality of light reception units.

### BACKGROUND

Near-infrared spectroscopy (NIRS) is a method for indirectly analyzing the activity occurring in a body part (e.g., a brain, a muscle, or other body part) of a person by measuring the degree of attenuation of near-infrared light (due to scattering and absorption by oxidized or non-oxidized hemoglobin) which varies with hemodynamic changes (e.g., changes in concentrations of oxidized and non-oxidized hemoglobin) due to the activity of the body part. More specifically, when hemodynamic changes due to the neural activity occurring in a brain is monitored, for example, near-infrared light having a wavelength range of about 630 nm to 1300 nm may be transmitted through a skull of the person to the depth of about 1 cm to 3 cm from the skull. By irradiating such near-infrared light to a head part of the person and detecting near-infrared light reflected or scattered therefrom, it is possible to monitor hemodynamic changes (e.g., a change in a concentration of blood oxygen (i.e., oxidized hemoglobin)) occurring in the cerebral cortex of the person. According to the recently introduced near-infrared spectroscopy, the neural activity occurring in a human brain (particularly, a cortex) may be quantified by arranging near-infrared light irradiation modules (i.e., light emission units) and near-infrared light detection modules (i.e., light reception units) at predetermined intervals in various parts of a head of a person, and analyzing signals related to hemodynamics (e.g., optical density (OD) signals based on the near-infrared spectroscopy) specified from optical signals generated by the light emission units and detected by the light reception units.

A monitoring device using an optical signal such as near-infrared light should include a plurality of light emission units and a plurality of light reception units arranged at predetermined intervals for accurate measurement, and should make close contact with a specific body part (e.g., a head, a muscle, or other body part) of a subject. Thus, the monitoring device is inevitably subject to the physical constraints that the plurality of light emission units and the plurality of light reception units should be disposed (i.e., a plurality of measurement channels should be implemented) within a limited space or area. Accordingly, there is a need to develop a monitoring device capable of implementing high-density measurement channels within a limited space or area.

In this connection, the inventor(s) present a technique for controlling a monitoring device capable of implementing a plurality of measurement channels at high density, by causing a plurality of light emission units to sequentially generate optical signals in a mutually exclusive manner according to a time division scheme, and dynamically controlling a measurement circuit gain of a light reception unit that detects an optical signal to be measured, which is generated by a light emission unit, on the basis of a distance between the light emission unit and the light reception unit.

### SUMMARY OF THE INVENTION

One object of the present invention is to solve all the above-described problems.

Another object of the invention is to provide a method, system, and non-transitory computer-readable recording medium for controlling a monitoring device including a plurality of light emission units (or sources) and a plurality of light reception units (or detectors) to implement a plurality of measurement channels at high density within a limited space or area in the monitoring device, by causing the plurality of light emission units to sequentially generate optical signals in a mutually exclusive manner according to a time division scheme; and with reference to a distance between an m-th light emission unit of the plurality of light emission units that generates an optical signal to be measured and an n-th light reception unit of the plurality of light reception units that detects the optical signal to be measured, dynamically controlling a measurement circuit gain that the n-th light reception unit uses to detect the optical signal generated by the m-th light emission unit.

The representative configurations of the invention to achieve the above objects are described below.

According to one aspect of the invention, there is provided a method for controlling a monitoring device including a plurality of light emission units (or sources) and a plurality of light reception units (or detectors), comprising the steps of: causing the plurality of light emission units to sequentially generate optical signals in a mutually exclusive manner according to a time division scheme; and with reference to a distance between an m-th light emission unit of the plurality of light emission units that generates an optical signal to be measured and an n-th light reception unit of the plurality of light reception units that detects the optical signal to be measured, dynamically controlling a measurement circuit gain that the n-th light reception unit uses to detect the optical signal generated by the m-th light emission unit.

According to another aspect of the invention, there is provided a system for controlling a monitoring device including a plurality of light emission units (or sources) and a plurality of light reception units (or detectors), comprising: a light emission management unit configured to cause the plurality of light emission units to sequentially generate optical signals in a mutually exclusive manner according to a time division scheme; and a light reception management unit configured to, with reference to a distance between an m-th light emission unit of the plurality of light emission units that generates an optical signal to be measured and an n-th light reception unit of the plurality of light reception units that detects the optical signal to be measured, dynamically control a measurement circuit gain that the n-th light reception unit uses to detect the optical signal generated by the m-th light emission unit.

In addition, there are further provided other methods and systems to implement the m invention, as well as non-transitory computer-readable recording media having stored thereon computer programs for executing the methods.

According to the invention, a plurality of light emission units included in a monitoring device may sequentially generate optical signals in a mutually exclusive manner according to a time division scheme, so that a plurality of optical signals respectively generated from the plurality of light emission units do not interfere with each other.

According to the invention, a measurement circuit gain that a light reception unit uses to detect an optical signal generated by a light emission unit may be dynamically controlled with reference to a distance between the light emission unit and the light reception unit, so that an optical signal generated at a farther location and an optical signal generated at a nearer location may be detected with the same level of strength.

According to the invention, even though each pair of a light emission unit and a light reception unit has a different optical signal transmission distance therebetween, the signal strength of an optical signal detected by a light reception unit may be maintained at the same level for all pairs, so that the influence of the diverse distances between the light emission units and light reception units may be minimized and a plurality of effective measurement channels (defined in correspondence to the pairs of the light emission units and light reception units) may be implemented within a limited space or area.

According to the invention, it is possible to implement various distance combinations of measurement channels that are defined by a plurality of light emission units and a plurality of light reception units arranged at predetermined intervals, thereby improving accuracy and reliability in estimating (or calculating) voxel-specific (or depth-specific) light absorption characteristics using a diffuse optical tomography technique.

### BRIEF DESCRIPTION OF THE DRAWINGS

FIG. 1A illustratively shows the external configuration of a monitoring device according to one embodiment of the invention.

FIG. 1B illustratively shows the external configuration of a monitoring device according to one embodiment of the invention.

FIG. 2 illustratively shows the internal configuration of a monitoring system according to one embodiment of the invention.

FIG. 3 illustratively shows a subset including a plurality of light emission units and a plurality of light reception units according to one embodiment of the invention.

FIG. 4 illustratively shows how each of the plurality of light emission units included in the subset sequentially generates an optical signal and at least one of the plurality of light reception units detects the optical signal, according to one embodiment of the invention.

FIG. 5 illustratively shows how a measurement circuit gain of a light reception unit is dynamically controlled according to one embodiment of the invention.

FIG. 6A illustratively shows a plurality of measurement channels that may be defined in the subset of FIG. 3 according to one embodiment of the invention.

FIG. 6B illustratively shows a plurality of measurement channels that may be defined in the subset of FIG. 3 according to one embodiment of the invention.

FIG. 6C illustratively shows a plurality of measurement channels that may be defined in the subset of FIG. 3 according to one embodiment of the invention.

FIG. 6D illustratively shows a plurality of measurement channels that may be defined in the subset of FIG. 3 according to one embodiment of the invention.

FIG. 7 illustratively shows a situation in which an optical signal generated from a light emission unit included in a first subset is detected by a light reception unit included in a second subset disposed adjacent to the first subset, according to according to one embodiment of the invention.

FIG. 8A illustratively shows a configuration of a source-detector array of a monitoring device including two or more subsets and combinations of measurement channels therein according to one embodiment of the invention.

FIG. 8B illustratively shows a configuration of a source-detector array of a monitoring device including two or more subsets and combinations of measurement channels therein according to one embodiment of the invention.

FIG. 8C illustratively shows a configuration of a source-detector array of a monitoring device including two or more subsets and combinations of measurement channels therein according to one embodiment of the invention.

FIG. 8D illustratively shows a configuration of a source-detector array of a monitoring device including two or more subsets and combinations of measurement channels therein according to one embodiment of the invention.

FIG. 8E illustratively shows a configuration of a source-detector array of a monitoring device including two or more subsets and combinations of measurement channels therein according to one embodiment of the invention.

FIG. 9 illustratively shows a subset including a plurality of light emission units and a plurality of light reception units according to another embodiment of the invention.

FIG. 10 illustratively shows how each of the plurality of light emission units included in the subset sequentially generates an optical signal and at least one of the plurality of light reception units detects the optical signal, according to another embodiment of the invention.

FIG. 11 illustratively shows how a measurement circuit gain of a light reception unit is dynamically controlled according to another embodiment of the invention.

FIG. 12A illustratively shows a plurality of measurement channels that may be defined in the subset of FIG. 9 according to another embodiment of the invention.

FIG. 12B illustratively shows a plurality of measurement channels that may be defined in the subset of FIG. 9 according to another embodiment of the invention.

### DETAILED DESCRIPTION

In the following detailed description of the present invention, references are made to the accompanying drawings that show, by way of illustration, specific embodiments in which the invention may be practiced. These embodiments are described in sufficient detail to enable those skilled in the art to practice the invention. It is to be understood that the various embodiments of the invention, although different from each other, are not necessarily mutually exclusive. For example, specific shapes, structures and characteristics described herein may be implemented as modified from one embodiment to another without departing from the spirit and scope of the invention. Furthermore, it shall be understood that the positions or arrangements of individual elements within each of the disclosed embodiments may also be modified without departing from the spirit and scope of the invention. Therefore, the following detailed description is not to be taken in a limiting sense, and the scope of the invention, if properly described, is limited only by the appended claims together with all equivalents thereof. In the drawings, like reference numerals refer to the same or similar functions throughout the several views.

Hereinafter, preferred embodiments of the present invention will be described in detail with reference to the accompanying drawings to enable those skilled in the art to easily implement the invention.

Herein, hemodynamics to be monitored by the monitoring device and the monitoring system may include blood composition (e.g., oxyhemoglobin concentration, deoxyhemoglobin concentration, blood oxygen saturation, etc.), blood flow, blood volume, and hemodynamics by muscle depth.

Configuration of the Monitoring System

Hereinafter, the internal configuration of a monitoring device and a monitoring system crucial for implementing the invention and the functions of the respective components thereof will be discussed.

A monitoring device according to one embodiment of the invention may be worn on a body part (e.g., a head, a muscle, or other body part) of a subject, and may function to measure a signal from the subject and process or analyze the measured signal as will be described below, thereby monitoring the activity occurring in the body part of the subject (e.g., neural activity occurring in the brain or hemodynamic changes occurring in the muscle).

Specifically, the monitoring device according to one embodiment of the invention may include a plurality of light emission units (or sources) for irradiating near-infrared light to a body part of a subject, and a plurality of light reception units (or detectors) for detecting near-infrared light reflected, scattered, or transmitted from the body part of the subject (more specifically, from the venous blood therein). For example, optical density (OD) signals based on near-infrared spectroscopy may be measured by the plurality of light emission units and the plurality of light reception units included in the monitoring device according to one embodiment of the invention.

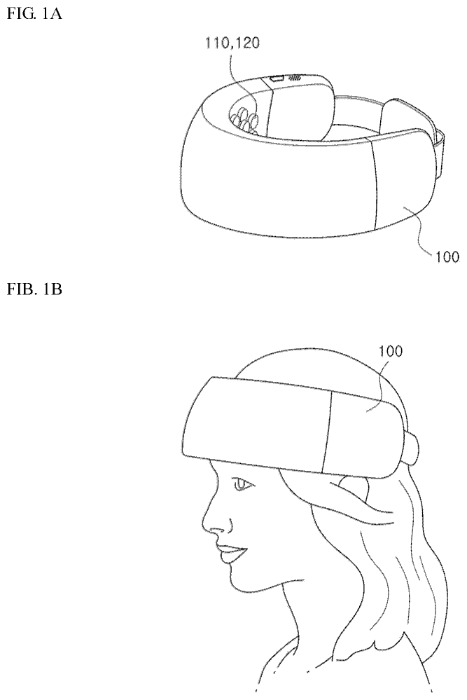

For example, the monitoring device according to one embodiment of the invention may be configured to be worn on a head part of the subject as shown in FIG. 1.

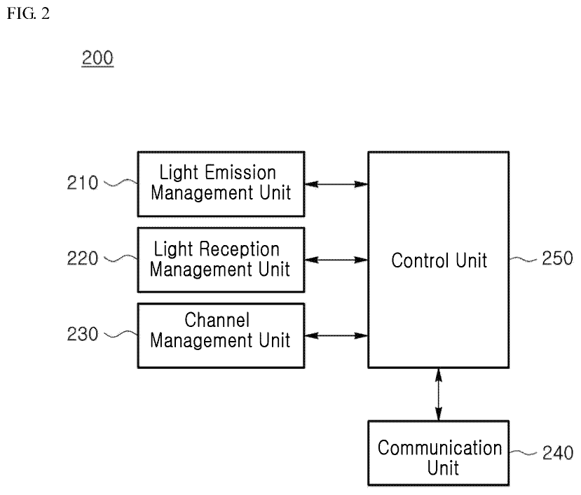

FIG. 2 illustratively shows the internal configuration of a monitoring system according to one embodiment of the invention.

Referring to FIG. 2, a monitoring system 200 according to one embodiment of the invention may comprise a light emission management unit 210, a light reception management unit 220, a channel management unit 230, a communication unit 240, and a control unit 250. According to one embodiment of the invention, at least some of the light emission management unit 210, the light reception management unit 220, the channel management unit 230, the communication unit 240, and the control unit 250 may be program modules to communicate with an external system (not shown). The program modules may be included in the monitoring system 200 in the form of operating systems, application program modules, and other program modules, while they may be physically stored in a variety of commonly known storage devices. Further, the program modules may also be stored in a remote storage device that may communicate with the monitoring system 200. Meanwhile, such program modules may include, but not limited to, routines, subroutines, programs, objects, components, data structures, and the like for performing specific tasks or executing specific abstract data types as will be described below in accordance with the invention.

Meanwhile, although the monitoring system 200 has been described as above, the above description is illustrative, and it will be apparent to those skilled in the art that at least a part of the components or functions of the monitoring system 200 may be implemented or included in the monitoring device (which is a portable device worn on a body part of the subject), as necessary. Further, in some cases, all the functions and components of the monitoring system 200 may be implemented or included in the monitoring device.

First, according to one embodiment of the invention, the light emission management unit 210 may function to cause the plurality of light emission units included in the monitoring device to generate optical signals for a body part of the subject.

Specifically, according to one embodiment of the invention, the light emission management unit 210 may cause the plurality of light emission units to sequentially generate optical signals in a mutually exclusive manner according to a time division scheme.

Next, according to one embodiment of the invention, the light reception management unit 220 may function to cause the plurality of light reception units included in the monitoring device to detect the optical signals generated by the plurality of light emission units with the distinction of the corresponding light emission units. The light reception management unit 220 according to one embodiment of the invention may function to cause the plurality of light reception units to detect the optical signals generated by the plurality of light emission units with the distinction of the corresponding light emission units, by dynamically controlling measurement circuit gains of the plurality of light reception units according to time intervals determined on the basis of the time division scheme applied to the plurality of light emission units.

Specifically, according to one embodiment of the invention, the light reception management unit 220 may function to, with reference to a distance between an m-th light emission unit of the plurality of light emission units and an n-th light reception unit of the plurality of light reception units, dynamically control a measurement circuit gain that the n-th light reception unit uses to detect the optical signal generated from the m-th light emission unit.

Further, according to one embodiment of the invention, the light reception management unit 220 may function to cause a light reception unit to only detect an optical signal generated by a light emission unit located within a predetermined distance from the light reception unit.

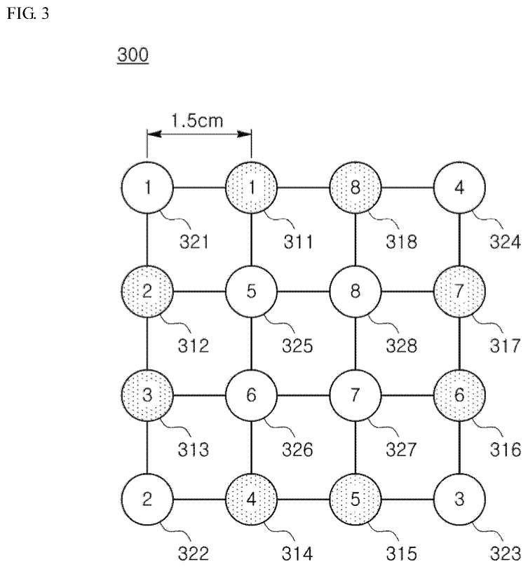

FIG. 3 illustratively shows a subset including a plurality of light emission units and a plurality of light reception units according to one embodiment of the invention.

According to one embodiment of the invention, in FIG. 3, a subset 300 may be formed by arranging eight light emission units 311 to 318 and eight light reception units 321 to 328 in a 4×4 grid pattern, and a grid spacing of the grid pattern array forming the subset 300 may be determined as 1.5 cm. However, the grid spacing according to the invention is not necessarily limited to 1.5 cm, and may be changed without limitation as long as the objects of the invention may be achieved.

Further, according to one embodiment of the invention, in FIG. 3, the eight light emission units 311 to 318 may sequentially generate optical signals in a mutually exclusive manner according to a time division scheme. This may prevent a near-far problem that may occur when two or more light emission units simultaneously generate optical signals (i.e., a problem that an optical signal generated at a farther location (i.e., having lower signal strength) cannot be measured due to an optical signal generated at a nearer location (i.e., having higher signal strength)).

In addition, according to one embodiment of the invention, in FIG. 3, measurement circuit gains of the eight light reception units 321 to 328 may be dynamically controlled on the basis of distances (e.g., 1.5 cm, about 2.12(=1.5×√2) cm, 3(=1.5×2) cm, about 3.35(=1.5×√5) cm, etc.) from a target light emission unit (any one of 311 to 318) that generates an optical signal to be measured. Further, according to one embodiment of the invention, in FIG. 3, each of the eight light reception units 321 to 328 may be controlled such that it does not detect an optical signal generated by a light emission unit located farther than about 3.35(=1.5×√5) cm from it (i.e., an optical signal having very low signal strength).

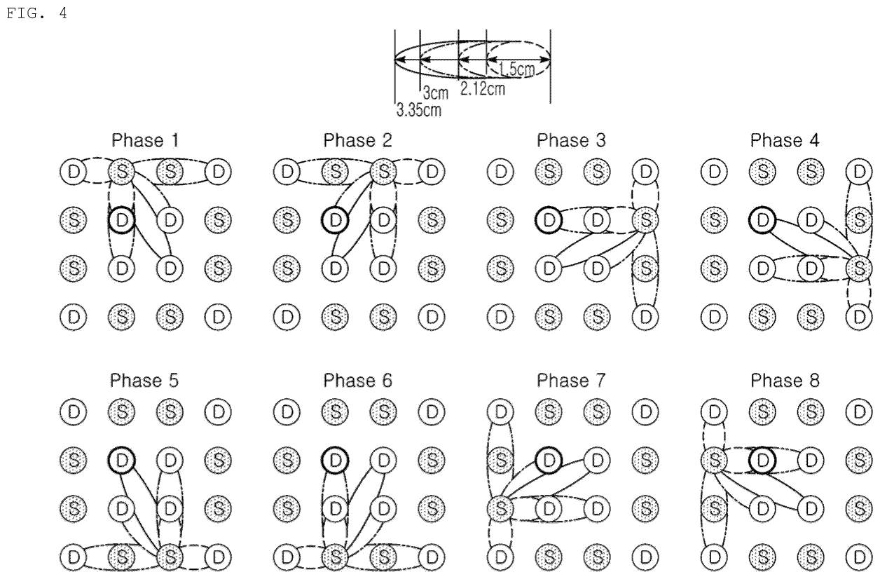

FIG. 4 illustratively shows how each of the plurality of light emission units included in the subset sequentially generates an optical signal and at least one of the plurality of light reception units (i.e., at least one light reception unit located within a predetermined distance from a target light emission unit) detects the optical signal, according to one embodiment of the invention.

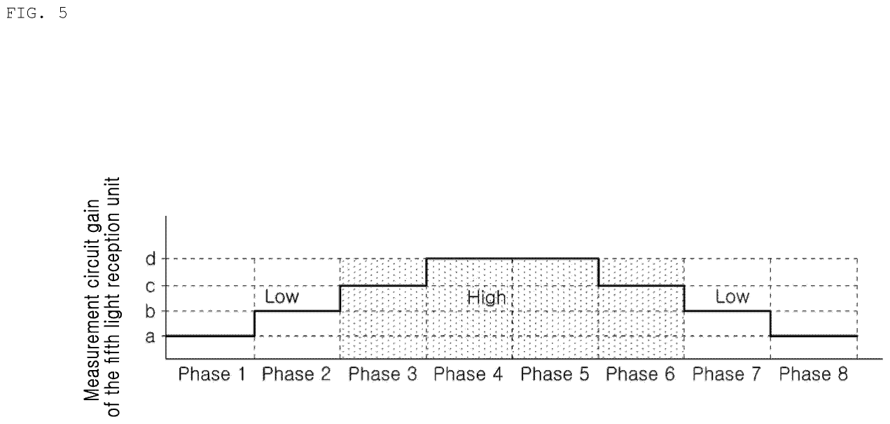

FIG. 5 illustratively shows how a measurement circuit gain of a light reception unit is dynamically controlled according to one embodiment of the invention.

In the embodiments of FIGS. 4 and 5, it will be discussed in detail how a measurement circuit gain of a light reception unit is dynamically controlled, with respect to the fifth light reception unit 325 in the subset 300.

Referring to FIGS. 4 and 5, when the first light emission unit 311 generates an optical signal and the other light emission units do not generate optical signals (i.e., in Phase 1), the light reception management unit 220 according to one embodiment invention may determine a measurement circuit gain of the fifth light reception unit 325, which detects the optical signal generated from the first light emission unit 311 located at 1.5 cm from the fifth light reception unit 325, to be “a”. That is, according to one embodiment of the invention, when the fifth light reception unit 325 detects an optical signal having the highest signal strength, the measurement circuit gain of the fifth light reception unit 325 may be determined to be the lowest value.

Referring further to FIGS. 4 and 5, when the eighth light emission unit 318 generates an optical signal and the other light emission units do not generate optical signals (i.e., in Phase 2), the light reception management unit 220 according to one embodiment invention may determine the measurement circuit gain of the fifth light reception unit 325, which detects the optical signal generated from the eighth light emission unit 318 located at about 2.12 cm from the fifth light reception unit 325, to be “b”. That is, according to one embodiment of the invention, when the fifth light reception unit 325 detects an optical signal having somewhat high signal strength, the measurement circuit gain of the fifth light reception unit 325 may be determined to be a somewhat low value.

Referring further to FIGS. 4 and 5, when the seventh light emission unit 317 generates an optical signal and the other light emission units do not generate optical signals (i.e., in Phase 3), the light reception management unit 220 according to one embodiment invention may determine the measurement circuit gain of the fifth light reception unit 325, which detects the optical signal generated from the seventh light emission unit 317 located at 3 cm from the fifth light reception unit 325, to be “c”. That is, according to one embodiment of the invention, when the fifth light reception unit 325 detects an optical signal having somewhat low signal strength, the measurement circuit gain of the fifth light reception unit 325 may be determined to be a somewhat high value.

Referring further to FIGS. 4 and 5, when the sixth light emission unit 316 generates an optical signal and the other light emission units do not generate optical signals (i.e., in Phase 4), the light reception management unit 220 according to one embodiment invention may determine the measurement circuit gain of the fifth light reception unit 325, which detects the optical signal generated from the sixth light emission unit 316 located at about 3.35 cm from the fifth light reception unit 325, to be “d”. That is, according to one embodiment of the invention, when the fifth light reception unit 325 detects an optical signal having the lowest signal strength, the measurement circuit gain of the fifth light reception unit 325 may be determined to be the highest value.

Next, according to one embodiment of the invention, the channel management unit 230 may function to manage a measurement channel defined in correspondence to a pair of any one of the plurality of light emission units and any one of the plurality of light reception units. According to one embodiment of the invention, an optical signal generated by a specific light emission unit disposed at a specific location in the monitoring device may be detected by a specific light reception unit disposed at a specific location in the monitoring device via a body part of the subject, and a channel (i.e., a path or an area) in which the optical signal is conveyed (i.e., propagated or transmitted) may be defined as the measurement channel.

Specifically, the channel management unit 230 according to one embodiment of the invention may define a measurement channel in correspondence to a pair of a specific light emission unit and a specific light reception unit located within a predetermined distance from each other. Further, the channel management unit 230 according to one embodiment of the invention may manage a plurality of measurement channels respectively defined in correspondence to a plurality of pairs as above, such that each of the plurality of measurement channels is distinguished in terms of a distance between the corresponding light emission unit and the corresponding light reception unit (i.e., in terms of signal strength of an optical signal generated by the corresponding light emission unit and detected by the corresponding light reception unit).

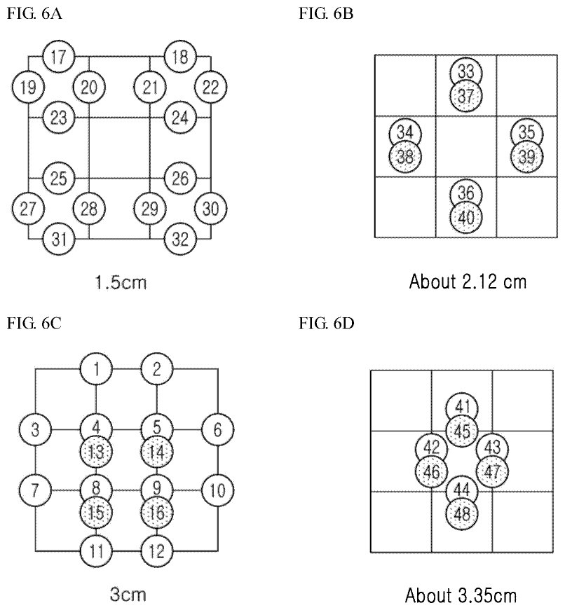

FIGS. 6A to 6D illustratively show a plurality of measurement channels that may be defined in the subset of FIG. 3 according to one embodiment of the invention.

It is noted that in FIGS. 6A to 6D, each of a plurality of circles marked with numbers indicates an individual measurement channel and is shown at a midpoint between a specific light emission unit and a specific light reception unit that are paired to define the corresponding measurement channel.

Referring to FIGS. 6A to 6D, various measurement channels may be defined for various distances in the subset of the 4×4 grid pattern shown in FIG. 3.

Specifically, according to one embodiment of the invention, in FIGS. 6A to 6D, a measurement channel may be defined only when a distance between a light emission unit and a light reception unit does not exceed about 3.35 cm (i.e., only when an optical signal having a significant level of signal strength may be detected by the light reception unit).

Further, according to one embodiment of the invention, in FIGS. 6A to 6D, a total of 16 measurement channels may be defined when a distance between a light emission unit and a light reception unit is 1.5 cm (see FIG. 6A); a total of 8 measurement channels may be defined when a distance between a light emission unit and a light reception unit is about 2.12 cm (see FIG. 6B); a total of 16 measurement channels may be defined when a distance between a light emission unit and a light reception unit is 3 cm (see FIG. 6C); and a total of 8 measurement channels may be defined when a distance between a light emission unit and a light reception unit is about 3.35 cm (see FIG. 6D).

Meanwhile, according to one embodiment of the invention, a plurality of subsets may be included in a single monitoring device if necessary, depending on the shape or area of a body part to be measured. In this case, optical signal interference may occur among the plurality of subsets, because an optical signal generated by a light emission unit included in a first subset may also be detected by a light reception unit included in a second subset adjacent to the first subset.

In order to prevent the optical signal interference among the plurality of subsets and increase the number of measurement channels per unit area (i.e., the density of measurement channels), the light emission management unit 210 according to one embodiment of the invention may modulate optical signals such that an optical signal generated by a light emission unit included in the first subset is orthogonal to an optical signal generated by a light emission unit included in the second subset. Specifically, the light emission management unit 210 according to one embodiment of the invention may modulate the optical signal generated by the light emission unit included in the first subset and the optical m signal generated by the light emission unit included in the second subset with codes that are orthogonal to each other.

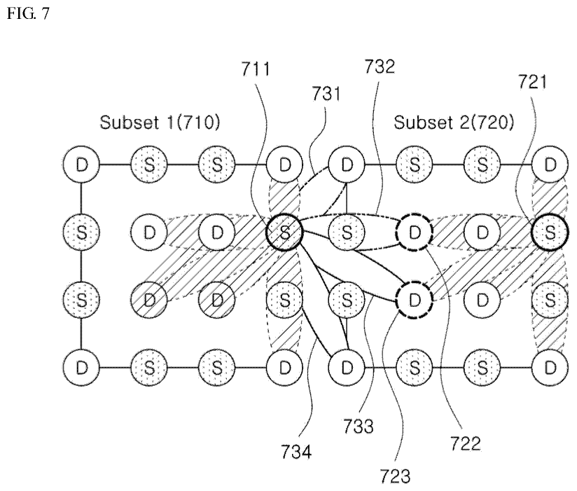

FIG. 7 illustratively shows a situation in which an optical signal generated from a light emission unit included in a first subset is detected by a light reception unit included in a second subset disposed adjacent to the first subset, according to according to one embodiment of the invention.

It is noted that in the embodiment of FIG. 7, a measurement channel is assumed to be defined only when a distance between a light emission unit and a light reception unit does not exceed about 3.35 cm.

Referring to FIG. 7, it may be assumed that a seventh light emission unit 711 in a first subset 710 and a seventh light emission unit 721 in a second subset 720 generate optical signals, respectively, as an operation corresponding to Phase 3 of FIG. 4. In this case, a fifth light reception unit 722 and a sixth light reception unit 723 in the second subset 720 may detect optical signals 732 and 733 generated by the seventh light emission unit 711 in the first subset 710, as well as optical signals 735 and 736 generated by the seventh light emission unit 721 in the second subset 720. Here, the monitoring system 200 according to one embodiment of the invention may modulate the optical signals 732 and 733 generated by the seventh light emission unit 711 in the first subset 710, and the optical signals 735 and 736 generated by the seventh light emission unit 721 in the second subset 720, with codes that are orthogonal to each other. Thus, the fifth light reception unit 722 and the sixth light reception unit 723 in the second subset 720 may accurately detect the above optical signals without interference, so that a new measurement channel may be defined between the first subset 710 and the second subset 720.

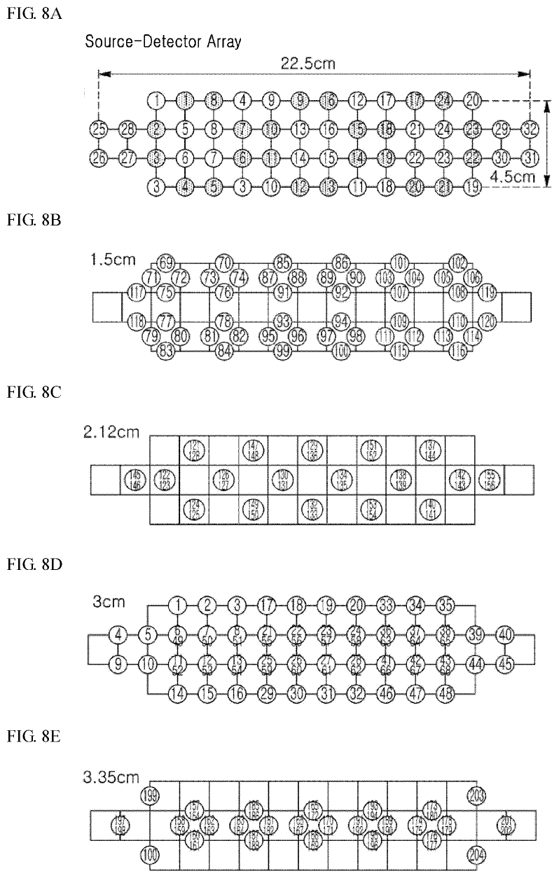

FIGS. 8A to 8E illustratively show a configuration of a source-detector array (or a light emission unit-light reception unit array) of a monitoring device including two or more subsets and combinations of measurement channels therein according to one embodiment of the invention.

Referring to FIG. 8A, a source-detector array included in a monitoring device according to one embodiment of the invention may be configured such that three subsets as shown in FIG. 3 are successively arranged and four light reception units are added to each of both ends thereof. Specifically, the source-detector array shown in FIG. 8 may include 24 light emission units and 32 light reception units, and may be sized at a width of 22.5 cm and a height of 4.5 cm.

Next, referring to FIGS. 8B to 8E, various measurement channels may be defined for various distances in the source-detector array. It is noted that in the embodiment of FIG. 8, a measurement channel is assumed to be defined only when a distance between a light emission unit and a light reception unit does not exceed about 3.35 cm (i.e., only when an optical signal having a significant level of signal strength may be detected by the light reception unit).

Referring further to FIGS. 8B to 8E, a total of 52 measurement channels may be defined when a distance between a light emission unit and a light reception unit is 1.5 cm (see FIG. 8B); a total of 36 measurement channels may be defined when a distance between a light emission unit and a light reception unit is about 2.12 cm (see FIG. 8C); a total of 68 measurement channels may be defined when a distance between a light emission unit and a light reception unit is 3 cm (see FIG. 8D); and a total of 48 measurement channels may be defined when a distance between a light emission unit and a light reception unit is about 3.35 cm (see FIG. 8E).

Meanwhile, the communication unit 240 according to one embodiment of the invention may function to enable the monitoring system 200 to communicate with an external device.

Lastly, the control unit 250 according to one embodiment of the invention may function to control data flow among the light emission management unit 210, the light reception management unit 220, the channel management unit 230, and the communication unit 240. That is, the control unit 250 may control inbound data flow or data flow among the respective components of the monitoring system 200, such that the light emission management unit 210, the light reception management unit 220, the channel management unit 230, and the communication unit 240 may carry out their particular functions, respectively.

Although the embodiments in which a source-detector array included in a monitoring device is configured to include subsets as shown in FIG. 3 have been mainly described above, it is noted that the subsets according to the invention are not necessarily limited as shown in FIG. 3, and may be changed without limitation as long as the objects of the invention may be achieved.

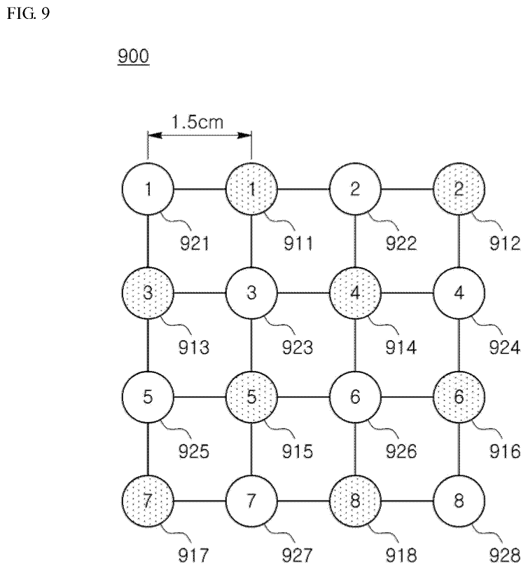

FIG. 9 illustratively shows a subset including a plurality of light emission units and a plurality of light reception units according to another embodiment of the invention.

According to another embodiment of the invention, in FIG. 9, a subset 900 may be formed by arranging eight light emission units 911 to 918 and eight light reception units 921 to 928 in a 4×4 grid pattern, and a grid spacing of the grid pattern array forming the subset 900 may be determined as 1.5 cm.

Further, according to another embodiment of the invention, in FIG. 9, the eight light emission units 911 to 918 may sequentially generate optical signals in a mutually exclusive manner according to a time division scheme. This may prevent a near-far problem that may occur when two or more light emission units simultaneously generate optical signals (i.e., a problem that an optical signal generated at a farther location (i.e., having lower signal strength) cannot be measured due to an optical signal generated at a nearer location (i.e., having higher signal strength)).

In addition, according to another embodiment of the invention, in FIG. 9, measurement circuit gains of the eight light reception units 921 to 928 may be dynamically controlled on the basis of distances (e.g., 1.5 cm, about 3.35(=1.5×√5) cm, etc.) from a target light emission unit (any one of 911 to 918) that generates an optical signal to be measured. Further, according to another embodiment of the invention, in FIG. 9, each of the eight light reception units 921 to 928 may not detect an optical signal generated by a light emission unit located farther than about 3.35 cm from it (i.e., an optical signal having very low signal strength).

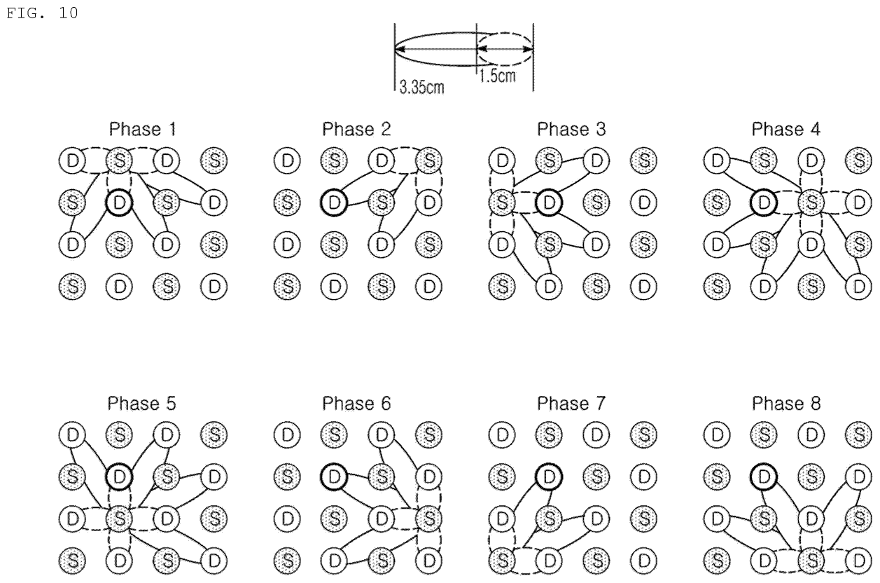

FIG. 10 illustratively shows how each of the plurality of light emission units included in the subset sequentially generates an optical signal and at least one of the plurality of light reception units detects the optical signal, according to another embodiment of the invention.

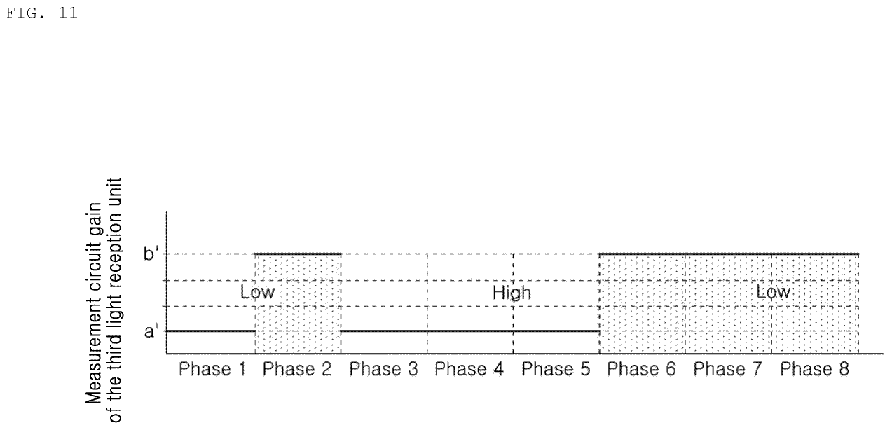

FIG. 11 illustratively shows how a measurement circuit gain of a light reception unit is dynamically controlled according to another embodiment of the invention.

In the embodiments of FIGS. 10 and 11, it will be discussed in detail how a measurement circuit gain of a light reception unit is dynamically controlled, with respect to the third light reception unit 923 in the subset 900.

Referring to FIGS. 10 and 11, when the first light emission unit 911 generates an optical signal and the other light emission units do not generate optical signals (i.e., in Phase 1), the light reception management unit 220 according to another embodiment invention may determine a measurement circuit gain of the third light reception unit 923, which detects the optical signal generated from the first light emission unit 911 located at 1.5 cm from the third light reception unit 923, to be “a”. That is, according to another embodiment of the invention, when the third light reception unit 923 detects an optical signal having the highest signal strength, the measurement circuit gain of the third light reception unit 923 may be determined to be the lowest value.

Referring further to FIGS. 10 and 11, when the second light emission unit 912 generates an optical signal and the other light emission units do not generate optical signals (i.e., in Phase 2), the light reception management unit 220 according to another embodiment invention may determine the measurement circuit gain of the third light reception unit 923, which detects the optical signal generated from the second light emission unit 912 located at about 3.35 cm from the third light reception unit 923, to be “b”. That is, according to another embodiment of the invention, when the third light reception unit 923 detects an optical signal having the lowest signal strength, the measurement circuit gain of the third light reception unit 923 may be determined to be the highest value.

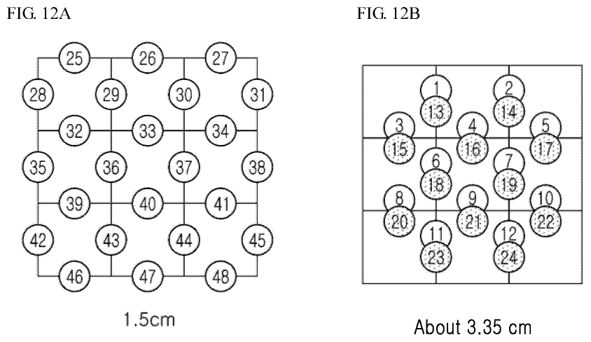

FIGS. 12A and 12B illustratively show a plurality of measurement channels that may be defined in the subset of FIG. 9 according to another embodiment of the invention.

It is noted that in FIGS. 12A and 12B, each of a plurality of circles marked with numbers indicates an individual measurement channel and is shown at a midpoint between a specific light emission unit and a specific light reception unit that are paired to define the corresponding measurement channel.

Referring to FIGS. 12A and 12B, various measurement channels may be defined for various distances in the subset of the 4×4 grid pattern shown in FIG. 9.

Specifically, according to another embodiment of the invention, in FIGS. 12A and 12B, a measurement channel may be defined only when a distance between a light emission unit and a light reception unit does not exceed about 3.35 cm (i.e., only when an optical signal having a significant level of signal strength may be detected by the light reception unit).

Further, according to another embodiment of the invention, in FIGS. 12A and 12B, a total of 24 measurement channels may be defined when a distance between a light emission unit and a light reception unit is 1.5 cm (see FIG. 12A), and a total of 24 measurement channels may be defined when a distance between a light emission unit and a light reception unit is about 3.35 cm (see FIG. 12B).

Although the cases where the measurement signal is an optical density signal based on near-infrared spectroscopy have been mainly described above, the measurement signal is not necessarily limited thereto, and it is noted that any other type of measurement signal may be assumed as long as the objects or effects of the methods, systems, and non-transitory computer-readable recording media described herein may be achieved.

Further, although the cases where a body part to be monitored is a head part (i.e., a brain) have been mainly described above, the body part to be monitored according to the invention is not necessarily limited thereto, and it is noted that any other body part (e.g., a muscle or other body part) that can be monitored on the basis of hemodynamics may be assumed to be the body part to be monitored according to the invention.

Meanwhile, according to one embodiment of the invention, a body part to be measured may be modeled as a heterogeneous space (i.e., a heterogeneous diffusion model) composed of a plurality of three-dimensional unit spaces (i.e., voxels) that may have various different light absorption characteristics. Further, according to one embodiment of the invention, light irradiated from a light emission unit in a monitoring device may be incident on all voxels constituting the body part to be measured, and light detected by a light reception unit after being transmitted through or reflected from certain voxels may include information on the corresponding voxels. Thus, according to one embodiment of the invention, an optical signal detected by a light reception unit may be formed as a sum of a plurality of unit optical signals that reflect the influence (or contribution) from each of the plurality of voxels, m and the light absorption characteristics of the body part to be measured may be identified per voxel (or depth) by using a diffuse optical tomography (DOT) technique for reconstructing the light absorption characteristics of each of the plurality of voxels constituting the body part to be measured (defined as the heterogeneous diffusion model) from a plurality of actual measurement signal values that are respectively measured by a plurality of light reception units included in the monitoring device.

In order to improve the reliability of the above diffuse optical tomography technique, measurement channels defined by a plurality of light emission units and a plurality of light reception units are required. According to the invention, it is possible to implement various distance combinations of measurement channels that are defined by a plurality of light emission units and a plurality of light reception units arranged at predetermined intervals, thereby achieving the distinctive effects of improving accuracy and reliability in estimating (or calculating) the voxel-specific (or depth-specific) light absorption characteristics using the diffuse optical tomography technique.

The embodiments according to the invention as described above may be implemented in the form of program instructions that can be executed by various computer components, and may be stored on a non-transitory computer-readable recording medium. The non-transitory computer-readable recording medium may include program instructions, data files, data structures and the like, separately or in combination. The program instructions stored on the non-transitory computer-readable recording medium may be specially designed and configured for the present invention, or may also be known and available to those skilled in the computer software field. Examples of the non-transitory computer-readable recording medium include the following: magnetic media such as hard disks, floppy disks and magnetic tapes; optical media such as compact disk-read only memory (CD-ROM) and digital versatile disks (DVDs); magneto-optical media such as floptical disks; and hardware devices such as read-only memory (ROM), random access memory (RAM) and flash memory, which are specially configured to store and execute program instructions. Examples of the program instructions include not only machine language codes created by a compiler or the like, but also high-level language codes that can be executed by a computer using an interpreter or the like. The above hardware devices may be configured to operate as one or more software modules to perform the processes of the present invention, and vice versa.

Although the present invention has been described above in terms of specific items such as detailed elements as well as the limited embodiments and the drawings, they are only provided to help more general understanding of the invention, and the present invention is not limited to the above embodiments. It will be appreciated by those skilled in the art to which the present invention pertains that various modifications and changes may be made from the above description.

Therefore, the spirit of the present invention shall not be limited to the above-described embodiments, and the entire scope of the appended claims and their equivalents will fall within the scope and spirit of the invention.

What is claimed is:

1. A method for controlling a monitoring device including a plurality of light emission units and a plurality of light reception units, comprising the steps of:
causing the plurality of light emission units to sequentially generate optical signals in a mutually exclusive manner according to a time division scheme; and
with reference to a distance between an m-th light emission unit of the plurality of light emission units that generates an optical signal to be measured and an n-th light reception unit of the plurality of light reception units that detects the optical signal to be measured, dynamically controlling a measurement circuit gain that the n-th light reception unit uses to detect the optical signal generated by the m-th light emission unit.

2. The method of claim 1, wherein the optical signals generated by the plurality of light emission units include near-infrared signals.

3. The method of claim 1, wherein each of the plurality of light reception units only detects an optical signal generated by a light emission unit located within a predetermined distance from the light reception unit.

4. The method of claim 1, wherein a measurement channel is defined in correspondence to a pair of a specific light emission unit and a specific light reception unit that are located within a predetermined distance from each other.

5. The method of claim 1, wherein the monitoring device includes at least one subset comprising at least one light emission unit and at least one light reception unit that are arranged in a pattern.

6. The method of claim 5, wherein an optical signal generated by a light emission unit included in a first subset and an optical signal generated by a light emission unit included in a second subset are modulated with codes that are orthogonal to each other.

7. The method of claim 1, wherein the plurality of light emission units and the plurality of light reception units are arranged in a grid pattern.

8. The method of claim 7, wherein each of the plurality of light emission units is arranged adjacent to at least one light reception unit, and each of the plurality of light reception units is arranged adjacent to at least one light emission unit.

9. A non-transitory computer-readable recording medium having stored thereon a computer program for executing the method of claim 1.

10. A system for controlling a monitoring device including a plurality of light emission units and a plurality of light reception units, comprising:
a light emission management unit configured to cause the plurality of light emission units to sequentially generate optical signals in a mutually exclusive manner according to a time division scheme; and
a light reception management unit configured to, with reference to a distance between an m-th light emission unit of the plurality of light emission units that generates an optical signal to be measured and an n-th light reception unit of the plurality of light reception units that detects the optical signal to be measured, dynamically control a measurement circuit gain that the n-th light reception unit uses to detect the optical signal generated by the m-th light emission unit.

11. The system of claim 10, wherein the optical signals generated by the plurality of light emission units include near-infrared signals.

12. The system of claim 10, wherein each of the plurality of light reception units only detects an optical signal generated by a light emission unit located within a predetermined distance from the light reception unit.

13. The system of claim 10, further comprising:
a channel management unit configured to define a measurement channel in correspondence to a pair of a specific light emission unit and a specific light reception unit that are located within a predetermined distance from each other.

14. The system of claim 10, wherein the monitoring device includes at least one subset comprising at least one light emission unit and at least one light reception unit that are arranged in a pattern.

15. The system of claim 14, wherein an optical signal generated by a light emission unit included in a first subset and an optical signal generated by a light emission unit included in a second subset are modulated with codes that are orthogonal to each other.

16. The system of claim 10, wherein the plurality of light emission units and the plurality of light reception units are arranged in a grid pattern.

17. The system of claim 16, wherein each of the plurality of light emission units is arranged adjacent to at least one light reception unit, and each of the plurality of light reception units is arranged adjacent to at least one light emission unit.
